(19) United States
(12) Patent Application Publication
(10) Pub. No.: US 2026/0041318 A1
(43) Pub. Date: Feb. 12, 2026

(54) Proctoscope and method for using a proctoscope

(71) Applicant: Primed Halberstadt Medizintechnik GmbH, Halberstadt (DE)

(72) Inventors: Sascha LEIBITZKI, Blankenburg (DE); Meik MALTZAHN, Halberstadt (DE); Peter TRÄGER, Halberstadt (DE)

(21) Appl. No.: 19/099,706

(22) PCT Filed: Jul. 25, 2023
(86) PCT No.: PCT/DE2023/000078

§ 371 Date: Jan. 29, 2025

(30) Foreign Application Priority Data
Feb. 5, 2022 (DE) 10 2022 002 849.2

(51) Int. Cl. A61B 1/31; A61B 1/00; A61B 1/32

(52) U.S. Cl. CPC A61B 1/31; A61B 1/00128; A61B 1/00177; A61B 1/32

(57) ABSTRACT

The invention, which relates to a proctoscope (1) and a method for using a proctoscope (1), is based on the object of providing an improved proctoscope (1) with which operative or surgical interventions in the area of the human rectum are made easier for the surgeon, whereby pain for the patient is reduced and whereby the proctoscope (1) is easy to handle and inexpensive to manufacture. This object is achieved in terms of the arrangement in that the fastening ring (2) has a plurality of position markers (10), a positioning aid (11) in the form of a 12 o'clock marking and a plurality of first locking elements (8), in that a second part is an operating tube (9) which can be arranged in an inner diameter of the fastening ring (2), which has a second locking element (17) which can be connected and fixed in a form-fitting manner to one of the first locking elements (8), which has a frustoconical or hemispherical, at least partially closed first end (12) and an surgical window (13), which is a rectangular opening arranged essentially along a cylinder wall of the operating tube (9), and in that a third part is an obturator (19) which can be introduced into the operating tube (9) and radially fixed, which has a first conical or hemispherical closed end (20) and which is inserted into the surgical tube (9) and fixed radially, completely closing the surgical window (13).

The invention relates to a proctoscope which is constructed in three parts and has as a first part a fastening ring which is open on its opposite sides and has a first region with an at least partially cylindrical shape or cylindrically conical shape and a flange-shaped second region.

The invention also relates to a method for using a proctoscope, wherein a three-part proctoscope is provided, wherein a first part is a fastening ring is open on its opposite sides and has a first region with an at least partially cylindrical shape or cylindrically conical shape and a flange-shaped second region, and wherein the proctoscope can be inserted into a rectum of a patient.

The content of this description is a proctoscope used in operative or surgical interventions in the area of the human rectum, which is also referred to as an anoscope or surgical proctoscope. This proctoscope is made up of several parts. It is used for the visual inspection of the rectum and as an aid in rectal interventions.

It is known that such proctoscopes or anoscopes are used to treat hemorrhoids, rectoceles and in the treatment of rectal mucosal prolapse. Proctoscopes or anoscopes are also used to perform mucopexy and recto-anal lifting as well as for direct tissue diagnostics. In particular, grade II-IV hemorrhoids are treated. Using a proctoscope or anoscope, surgeons are able to carry out minimally invasive operations such as mucopexy or recto-anal lifting that are relatively painless for the patient.

Various proctoscopes and anoscopes are known from the state of the art.

EP 3 310 246 B1 discloses an anoscope that can be used in the field of proctology as a diagnostic and/or surgical instrument. The problem to be solved is to improve an anoscope that can be used in the surgical and diagnostic field.

To solve this problem, an anoscope is provided which comprises a movable part which rotates within a fixed part and is provided with a window.

US 2008/0275306 A1 generally discloses an anoscope for anorectal diagnostics and surgery and in particular a composite anoscope consisting of three frustoconical components that are to be inserted into one another. The problem to be solved is to provide a composite anoscope that is free of the disadvantages known from the prior art. These disadvantages are that known anoscopes neither allow an exact assessment of the circumferential extent of the threads nor a determination of the distance of the threads from the anal margin. In addition, no fixation of the anoscope to the anal tissue is provided during the operation.

The anoscope according to the invention offers the advantage that its three components, when introduced into the anal canal, form a single frustoconical body with a pointed cone tip and a compact and smooth outer surface, which is suitable for penetrating the anal canal very quickly and in an atraumatic manner.

WO 2004/021874 A1 relates to an anoscope that can be used in proctology as a diagnostic and/or surgical instrument. The aim is to overcome the disadvantage that the surgical window only opens onto a fixed area of the speculum. This is intended to improve an anoscope that can be used in surgical practice.

To achieve this, there is provided an anoscope comprising a first hollow body component open at opposite ends and a second hollow body component open at opposite ends, formatively coupled to the first hollow body component and arranged to rotate coaxially within, the first hollow body component and the second hollow body component being provided with a window arranged to make a portion of the rectal mucosa accessible, the window having a dimension and a shape to enable use of surgical means.

WO 002021021396 discloses an anoscope comprising an elongated hollow member having a longitudinal slot configured to receive tissue within an interior of the hollow member. The device also includes a clamp applicator. The clamp applicator has a body slidably received in the slot and jaws at a distal end of the body configured to hold a ligature clamp. The clamp applicator further comprises a piston slidably coupled to the body configured to actuate the jaws. When the clamp applicator is inserted into the slot and the piston is moved relative to the body, the jaws close and deform the ligature clamp from an open position to a closed position around the tissue received in the slot.

These state-of-the-art proctoscopes do not have a so-called 12-hour position for a rectocele, i.e. a bulge in the lower rectum above the anal sphincter. One advantage of such a 12-hour position is that it enables rectocele treatment.

Furthermore, the known solutions do not have a thread attachment by means of which a surgical thread can be attached to a part of the proctoscope. Such a possibility of attaching a thread has the advantage that these attachments can be fixed during the operation in such a way that the surgeon's field of vision and work is not impaired.

In addition, unwanted rotations of the proctoscope cannot be effectively prevented, which can lead to torsion of the patient's tissue with the corresponding adverse effects.

In addition, localization of the dentate line is difficult using the known solutions. The dentate line marks the transition between the insensitive mucous membrane of the rectum and the highly sensitive squamous epithelium of the anal canal and represents an important orientation aid in the anal canal.

There is therefore a need for an improvement of the known state of the art and thus for an improved proctoscope for operative or surgical interventions in the area of the human rectum.

The object of the invention is to provide an improved proctoscope with which operative or surgical interventions in the area of the human rectum are made easier for the surgeon, whereby pain for the patient is reduced and whereby the proctoscope is easy to handle and inexpensive to manufacture.

In particular, ease of handling by the surgeon should be ensured and the overview of the surgical area should be improved.

The solution is a three-part proctoscope, which consists of the components fastening ring, surgical tube and obturator.

The fastening ring, as the first part of the proctoscope, has a first region with an at least partially cylindrical shape or cylindrically conical shape and a flange-shaped second region. The fastening ring is hollow and open on the opposite sides or ends. The first side is located on the flange-shaped second region and the opposite side is located on an end of the at least partially cylindrical or cylindrically conical first region facing away from the flange-shaped second region.

The fastening ring is also designed to be transparent to allow the localization of the dentate line.

The flange-shaped second area of the fastening ring is connected to a handle, which can be provided with a grip recess, for example for the thumb and index finger. The flange-shaped second area of the fastening ring also has several so-called fastening holes. Using these fastening holes, it is possible for a surgeon, for example, to sew the fastening ring into place for the duration of a perianal intervention using several skin sutures in order to ensure that the proctoscope is held securely in the area of the patient's rectum.

The fastening ring preferably has in its cylindrical first region a plurality of inwardly directed L-shaped first locking elements. With these first locking elements, a so-called surgical tube can be temporarily fixedly connected or locked to the fastening ring, whereby only one L-shaped first locking element is used for locking. Each of the L-shaped first locking elements allows the surgical tube to be locked in the fastening ring in a different position or the surgical tube to be oriented relative to the fastening ring.

For this purpose, the surgical tube with the obturator inserted into the surgical tube is pushed into the fastening ring and locked or fixed into the first locking elements by a rotating movement that may only be a few degrees. The obturator simultaneously serves to push the tissue back out of the surgical window of the surgical tube when the proctoscope is inserted and as a handle or as an extension of the grip surface for releasing, rotating and locking the surgical tube.

The fastening ring also has several position markers which are arranged at equal intervals along the circumference of the flange-shaped area of the fastening ring. Such position markers show a surgeon the orientation of a so-called surgical window of the proctoscope, in which by using suitable instruments an operative or surgical procedure can be carried out in the area of the human rectum by the surgeon.

In addition to these positioning marks, a positioning aid in the form of a 12 o'clock mark is provided, which points upwards on the human body in the direction of the patient's spine or head, when the fastening ring is positioned accordingly in the rectum of a patient.

A second part of the proctoscope is the surgical tube. The surgical tube has a cylindrical shape or a cylindrical conical shape and is designed as an elongated, hollow body. The first end of the surgical tube has the shape of a truncated cone or hemisphere, is partially closed, with an essentially rectangular opening arranged essentially laterally or essentially along a cylinder wall of the surgical tube, which is referred to as the surgical window, being provided at this end. It is also intended that the essentially rectangular opening of the surgical window partially protrudes into the truncated cone-shaped or hemispherical first end of the surgical tube.

The surgical window extends, for example, over a third of the total length of the surgical tube, starting at the first end of the surgical tube. The surgical window is designed to be almost rectangular and is dimensioned in such a way that a surgeon can perform a surgical procedure on the patient's tissue protruding in the surgical window. In an exemplary embodiment, the surgical window has a length of approximately 4 to 6 cm and a width of approximately 2 to 3 cm.

The surgical tube is designed with an opening at its second end disposed opposite the first end. This opening essentially corresponds to the diameter of the cylindrical surgical tube at the second end. This opening can also be designed to be slightly larger than the diameter of the cylindrical surgical tube at the second end.

At this second end, the surgical tube is designed, for example, with corresponding holes or dimples along the circumference of the second end of the surgical tube so that it can be securely gripped and held by a surgeon. Furthermore, at least one first alignment marker is arranged at this second end, which can be provided with a thread attachment.

A second locking element is arranged at the second end of the surgical tube to temporarily lock the surgical tube to the fastening ring. This second locking element is designed in such a way that it can be snapped into the first locking elements of the fastening ring. Such temporary locking can be achieved with a so-called bayonet lock, which is known to enable a mechanical connection between two cylindrical parts along their longitudinal axis that can be quickly established and released.

It is also intended that a radial fixation is arranged at the second end of the surgical tube to create a positive, locking connection with another radial fixation formed on the obturator. The obturator is thus introduced into the surgical tube and connected to it in a positive manner or fixed to it in a locking manner.

It is also intended that the part of the system that can rotate in the fastening ring, i.e. the surgical tube, can be locked and thus fixed by rotation in, for example, 7 positions. These positions are preferably 1 o'clock, 3 o'clock, 5 o'clock, 7 o'clock, 9 o'clock, 11 o'clock and 12 o'clock position, which are known, for example, from an analog clock face. These positions are indicated accordingly using the position markers and the 12 o'clock marking on the fastening ring. In the event that the first alignment marker of the surgical tube is aligned with the 3 o'clock position marker on the fastening ring, the surgical window of the surgical tube is arranged in the 3 o'clock direction in the patient's rectum. The surgeon can therefore carry out a surgical procedure in this 3 o'clock direction. These relationships also apply to the other selectable positioning options at the positions 1 o'clock, 3 o'clock, 5 o'clock, 7 o'clock, 9 o'clock, 11 o'clock and 12 o'clock.

The third part of the three-part proctoscope is the obturator, which has a substantially cylindrical or cylindrically conical elongated shape. The obturator has at its first end a conical or hemispherical closure. This closure is designed for insertion into the surgical tube and thus completely closes the surgical window of the surgical tube. For this purpose, the outer diameter of the obturator is optimally adapted to the inner diameter of the surgical tube. For this purpose, the second end of the obturator is also optimally adapted to the second end of the surgical tube with its conical or hemispherical shape.

The second end of the obturator is designed, for example, with corresponding holes or dimples along the circumference of the second end of the obturator, thus allowing it to be securely grasped and held by a surgeon.

In addition, the second end of the obturator has a radial fixation with which the obturator can be positively connected to the surgical tube. In addition, the second end of the obturator has a second alignment marker.

The obturator is inserted into the surgical tube in such a way that the first alignment marker of the surgical tube, which can also be a suture attachment, corresponds to the second alignment marker of the inserted obturator.

Completely closing the surgical window of the surgical tube with the obturator enables improved, friction-reduced insertion of the proctoscope, while stretching the muscular wall of the rectum but not damaging the mucous membrane.

In addition, the surgical window of the surgical tube can be rotated safely to different positions when the obturator is inserted into the surgical tube. After rotation to a desired position, the obturator is removed from the surgical tube, thus exposing the surgical window of the surgical tube to the surgeon. Once the surgical procedure has been carried out in this position, the obturator can be reinserted into the surgical tube, locked, and the surgical window can be closed. The surgical tube is then rotated with the inserted and locked obturator to a subsequent position desired by the surgeon. Once the subsequent desired position has been reached, the obturator is removed from the surgical tube, thus exposing the surgical window of the surgical tube to the surgeon in the desired position.

By making the fastening ring out of a transparent material, the dentate line can be localized, after the surgical tube with the obturator has been removed from the fastening ring.

It is intended that the surgical tube can be fixed in the fixing ring at at least 7 positions (1 o'clock, 3 o'clock, 5 o'clock, 7 o'clock, 9 o'clock, 11 o'clock and 12 o'clock), with the 12 o'clock position being intended for rectocele procedures.

According to the invention, it is also provided that the further radial fixation is U-shaped, with arcuate locking protrusions being arranged on opposite parallel outer legs of the U-shaped further radial fixation, that the radial fixation is also U-shaped, with arcuate locking recesses being arranged on opposite parallel inner legs of the radial fixation, and that the arcuate locking protrusions of the further radial fixation and the arcuate locking recesses of the radial fixation have corresponding, complementary shapes and latch with one another in a form-fitting manner when the obturator is inserted into the surgical tube.

The further radial fixation is designed as a protrusion on the obturator with a U-shaped outer contour and, for example, two arched locking protrusions arranged opposite one another on the parallel outer legs of the U-shaped further radial fixation.

The radial fixation is designed as a notch in the surgical tube with a U-shaped inner contour and, for example, two arcuate locking recesses arranged opposite one another on the parallel inner legs of the U-shaped radial fixation.

Due to the inventive design of the arcuate locking protrusions of the further radial fixation corresponding to the arcuate locking recesses of the radial fixation, the obturator can be inserted into the surgical tube when the orientation of the radial fixation of the surgical tube matches the orientation of the further radial fixation of the obturator. This insertion of the obturator into the surgical tube is continued until the arcuate locking protrusions of the further radial fixation have reached the position of the arcuate locking recesses of the radial fixation and these engage with one another due to their complementary shapes, thus creating a positive connection.

According to the invention, it is further provided that at least the obturator and the surgical tube are made of a material that can be used in medical technology, laboratory technology and in the diagnostic field. Polypropylene homopolymers, such as Purell HP 373, Purell HP 371, PPH 9020 or PPM H250, are known for such areas of application. The material from which the obturator and the surgical tube are made should have a ball indentation hardness according to ISO 2039-1 of 55 to 70 MPa and a tensile modulus according to ISO 527-1,-2 of 1200 to 1700 MPa, i.e. have sufficient strength and be at least partially elastically deformable.

The deformability is particularly advantageous in the region of the radial fixation and the further radial fixation, since the further radial fixation can thus be inserted into the radial fixation, although the outer dimension of the further radial fixation, i.e. the maximum distance between the arcuate locking protrusions, is greater than the inner dimension of the radial fixation of the surgical tube in the region of the parallel inner legs, with the exception of the region of the arcuate locking recesses.

In preparation for the use of the proctoscope 1 in the rectum of a human, the proctoscope, consisting of its fastening ring, the surgical tube and the obturator, is assembled in a first step, whereby the parts are connected or fixed to each other accordingly.

For this purpose, the obturator is inserted into the surgical tube, whereby it is important to ensure that the further radial fixation of the obturator (nose of the obturator) completely engages or is positively connected to the radial fixation of the surgical tube (groove of the surgical tube) and that the obturator is completely inserted into the surgical tube.

According to the invention, this connection between the obturator and the surgical tube form is formed by providing the further radial fixation in a U-shape, wherein arcuate locking protrusions are provided on opposite parallel outer legs of the U-shaped further radial fixation, by providing the radial fixation in a U-shape, wherein arcuate locking recesses are provided on opposite parallel inner legs of the radial fixation, wherein the arcuate locking protrusions and the arcuate locking recesses have corresponding, complementary shapes and engage with one another when the obturator is inserted into the surgical tube and produce a positive connection.

The surgical tube with the obturator inserted is then inserted into the fastening ring and the second locking element of the surgical tube is connected or fixed in a form-fitting manner to any of the seven first locking elements (bayonet locks) of the fastening ring. The surgical tube with the obturator inserted is thus fixed and locked in the fastening ring.

After the proctoscope has been lubricated, it can be inserted into the patient. It is important to ensure that the handle of the fastening ring is pointing at 6 o'clock. It is helpful to pull both buttocks apart to insert the proctoscope as deeply as possible into the anal canal and rectum. This will ensure that the fastening ring is securely and correctly positioned during the procedure.

After the proctoscope has been inserted, it is fixed in place using skin sutures through the holes at the 12 o'clock, 5 o'clock and 7 o'clock positions.

To perform a surgical procedure, the obturator is then removed from the surgical tube which is arranged and fixed in the fastening ring.

The surgical window of the surgical tube is now exposed and the surgeon can perform a surgical procedure in the area of the human rectum. This can be done at the point described as the 12 o'clock position if the surgical window has been aligned accordingly. To this end, the first alignment marker is aligned with the positioning aid or the 12 o'clock mark.

To select a different direction or position for the surgical window, the obturator is reinserted into the surgical tube. The second alignment marker on the obturator is brought into alignment with the first alignment marker on the surgical tube.

This insertion of the obturator into the surgical tube again completely closes the surgical window in the surgical tube.

When the obturator is inserted into the surgical tube and the surgical window is closed, the surgical tube and thus the surgical window can be safely rotated to different positions. Such rotation or alignment of the surgical window can take place in all directions shown by the position markers on the fastening ring or the 12 o'clock mark.

To change the direction or position of the surgical window, the surgical tube must be unlocked with the obturator, pulled out completely and repositioned. Turning the surgical tube in the anal canal must be avoided at all costs, as this could lead to torsion of the tissue.

Depending on the indication, the surgical window of the surgical tube can be positioned at seven different locations, which are marked on the fastening ring as indentations (1 o'clock, 3 o'clock, 5 o'clock, 7 o'clock, 9 o'clock, 11 o'clock and 12 o'clock positions). The direction or position of the window opening of the surgical tube is marked extracorporeally via the first alignment marker, which also serves as a thread attachment.

To perform a surgical procedure, the obturator is then removed from the surgical tube arranged and fixed in the fastening ring, thus exposing the surgical window of the surgical tube at the new position indicated by the position markers or the 12 o'clock mark.

Before the proctoscope is removed from the rectum, the obturator can be reinserted into the surgical tube disposed in the fastening ring, locked, and the surgical window be closed. The three skin sutures are then severed and the proctoscope is removed. Alternatively, the fastening ring can be removed without the surgical tube and with the obturator inserted, after the three skin sutures have been severed.

The proctoscope according to the invention has in particular the following advantages:
easy, simple handling for the surgeon,
ergonomic design,
distinctive design elements,
a secure connection of the fastening ring with the surgical tube by way of a bayonet lock,
possibility for attaching the surgical thread to the surgical tube,
multiple alignment markers,
identification of the surgical positions,
a cost-effective, outpatient surgical procedure when using the proctoscope according to the invention.

The above-described features and advantages of this invention will be better understood and appreciated after careful study of the following detailed description of the preferred, non-limiting exemplary embodiments of the invention in conjunction with the accompanying drawings, which show in:

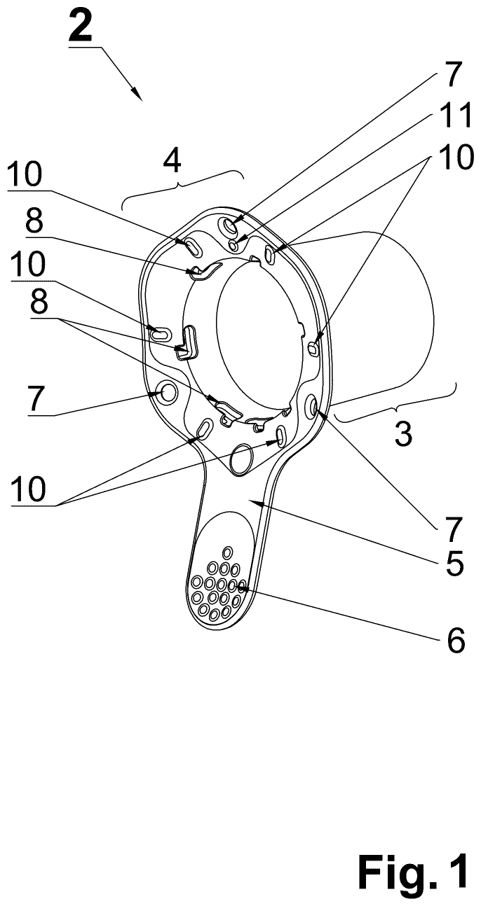

FIG. 1: the fastening ring of the proctoscope according to the invention,

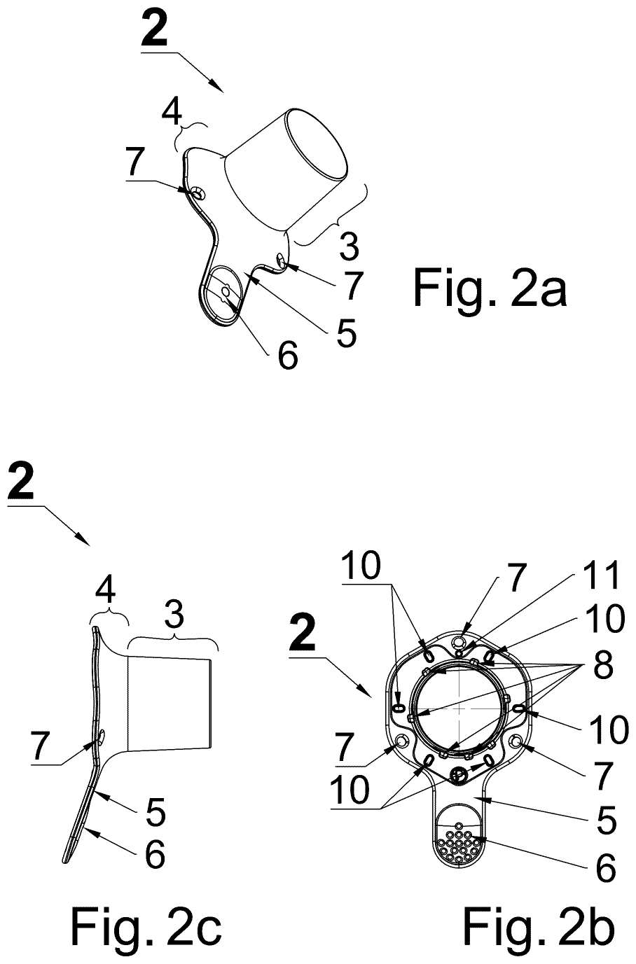

FIGS. 2a, 2b, 2c: further views of the fastening ring according to the invention,

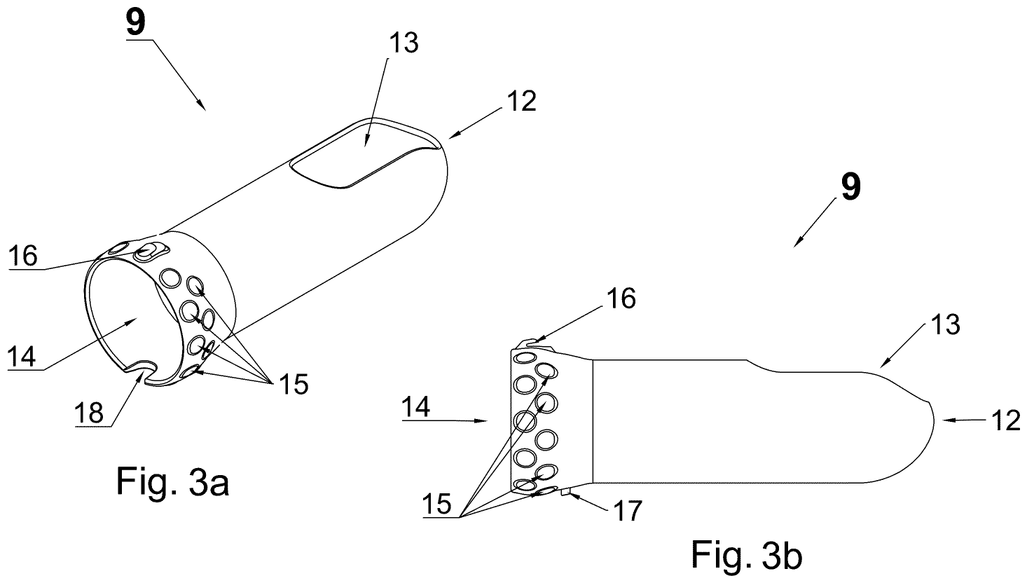

FIGS. 3a, 3b: the surgical tube of the proctoscope according to the invention,

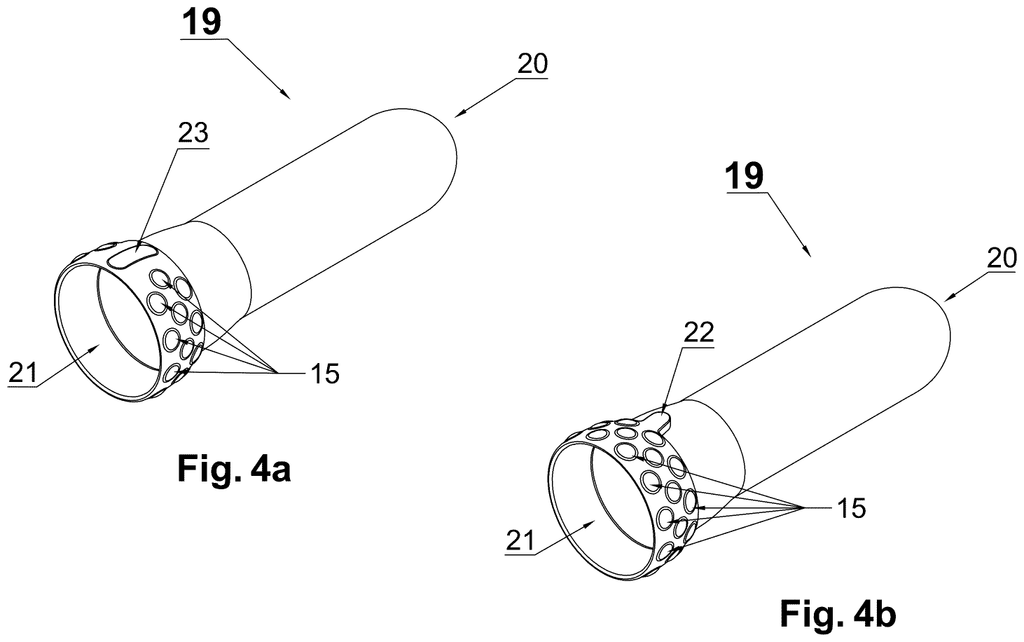

FIGS. 4a, 4b: the obturator of the proctoscope according to the invention,

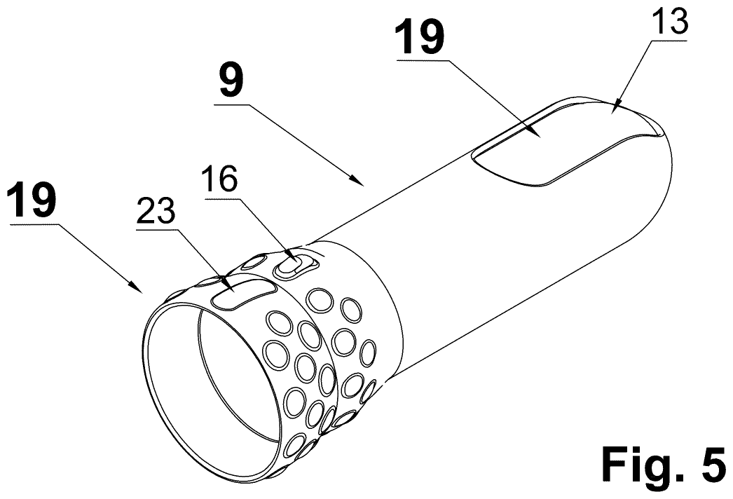

FIG. 5: the obturator inserted into the surgical tube,

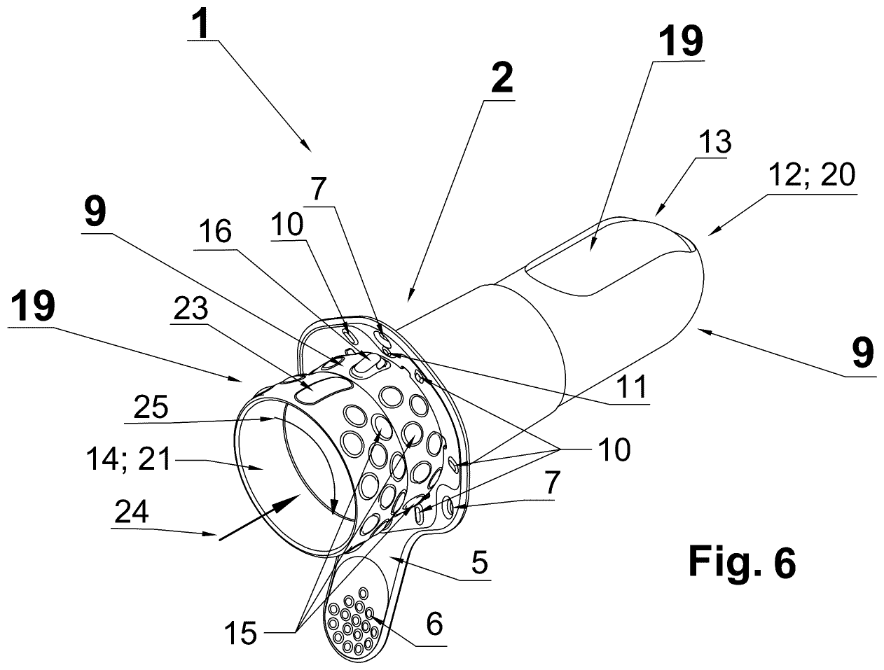

FIG. 6: the assembled proctoscope according to the invention,

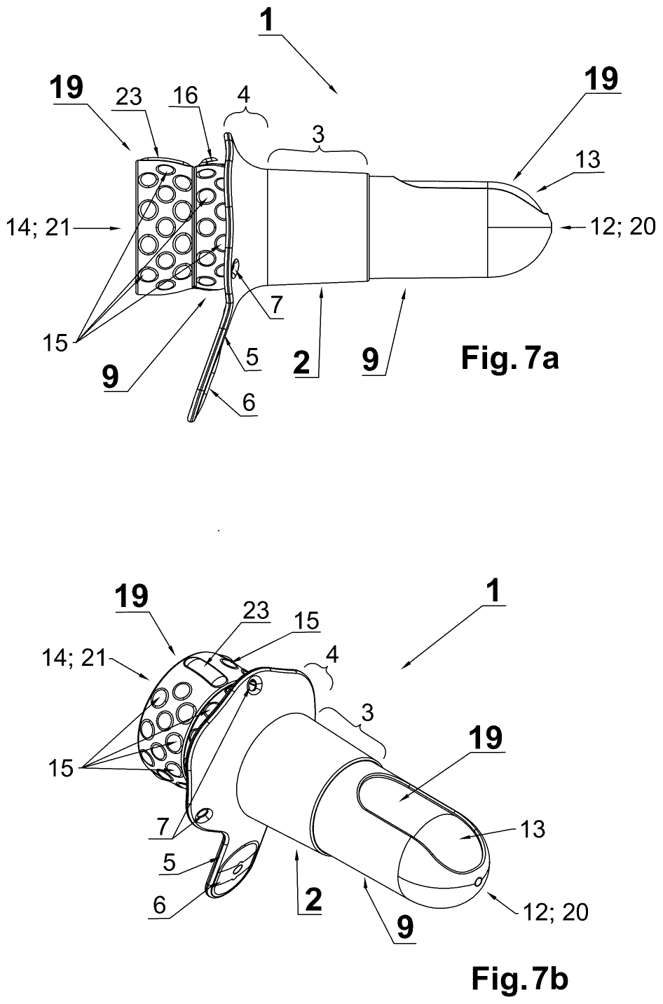

FIGS. 7a, 7b: further views of the proctoscope according to the invention,

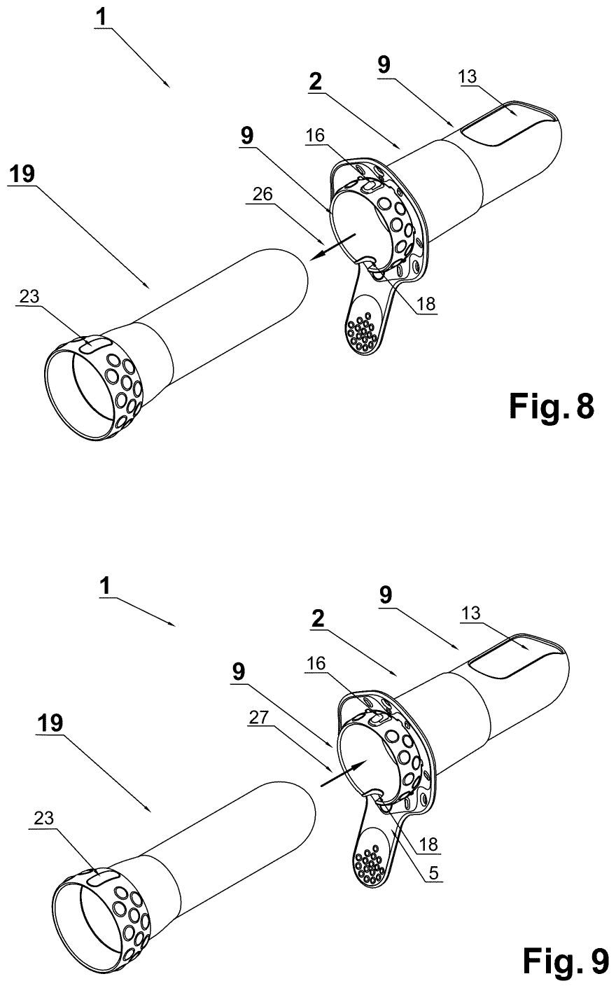

FIG. 8: removal of the obturator from the surgical tube,

FIG. 9: insertion of the obturator into the surgical tube,

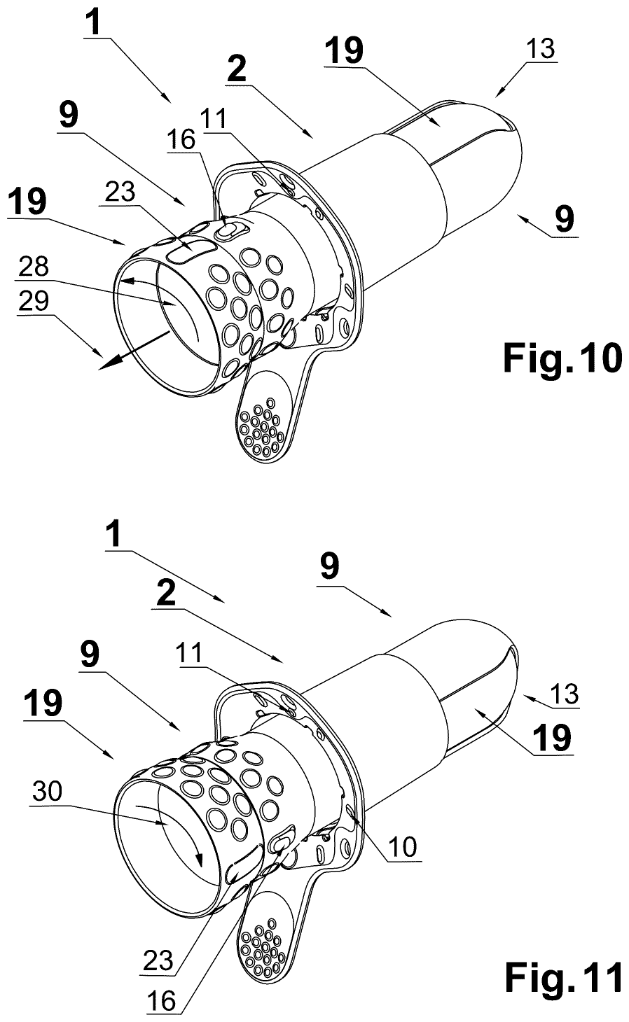

FIG. 10: release of the surgical tube with obturator from the fastening ring,

FIG. 11: rotation of the surgical tube with obturator in the fastening ring,

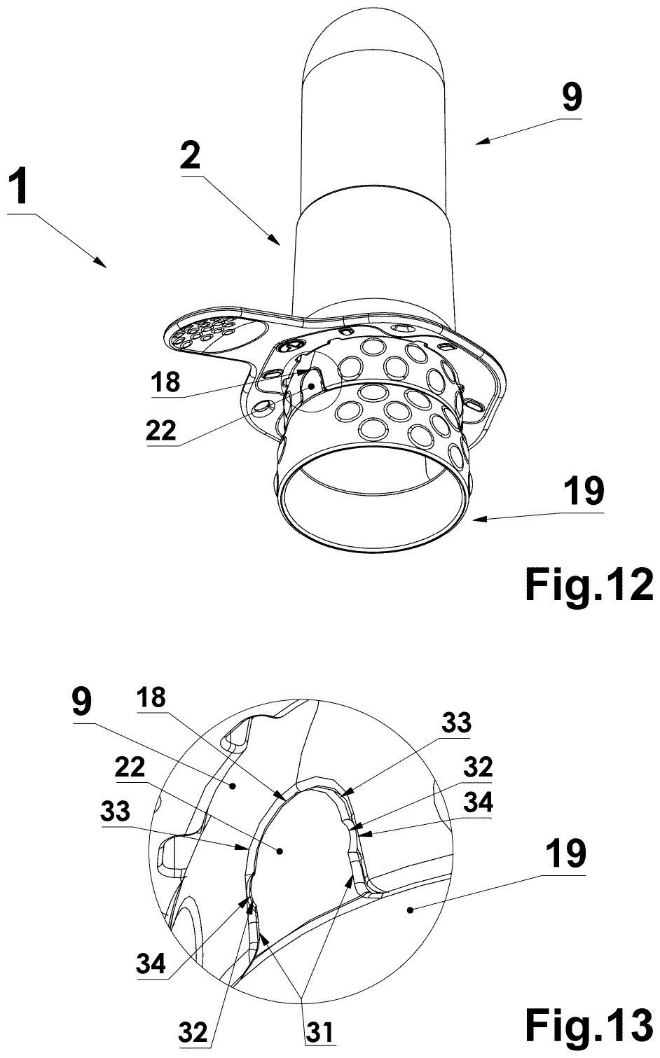

FIG. 12: the assembled proctoscope, and

FIG. 13: an enlargement of the area of radial fixation on the surgical tube and the further radial fixation on the obturator.

FIG. 1 shows a first part of the proctoscope 1 according to the invention, the fastening ring 2. The fastening ring 2 has a first region 3 with an at least partially cylindrical or cylindrically conical shape and a flange-shaped second region 4. The fastening ring 2 is hollow and open on the opposite sides or ends.

The flange-shaped second region 4 of the fastening ring 2 is connected to a handle 5, which is provided with a grip recess 6, for example for the thumb and index finger. The flange-shaped second area 4 of the fastening ring 2 has three fastening holes 7, as shown in FIG. 1. Using these fastening holes 7, the fastening ring 2 can be sewn perianally for the duration of an intervention using three skin sutures in order to ensure that the proctoscope is held securely in the area of the patient's rectum.

The fastening ring 2 has in its cylindrical first region 3 a plurality of inwardly directed L-shaped first locking elements 8. With these first locking elements 8, an operating tube 9 (not shown in FIG. 1) is temporarily firmly connected to the fastening ring 2 and locked. For this purpose, the operating tube 9 is pushed into the fastening ring 2 and locked into the first locking elements 8 by means of a rotational movement which may only be a few degrees, as will be shown later.

In addition, the fastening ring 2 has six position markers 10, which are arranged equidistally along the circumference of the flange-shaped second region 4 of the fastening ring 2. These position markers 10 are used to indicate the alignment of a so-called surgical window 10 in the surgical tube 9 of the proctoscope 1. The surgical tube 9 with the surgical window 10 are not shown in FIG. 1.

In FIG. 1, another positioning aid 11, a 12 o'clock mark, is shown in addition to the six position markers 10. This 12 o'clock position is intended in particular for rectocele procedures.

FIGS. 2A, 2B and 2C show further views of the fastening ring 2.

For a better understanding, the fastening ring 2 already shown in FIG. 1 is shown in FIG. 2a in a further perspective view, in FIG. 2b in a frontal view and in FIG. 2c in a side view. All elements shown in FIGS. 2a, 2b and 2c have already been described in detail in conjunction with FIG. 1, so that a repetition is not necessary at this point.

FIGS. 3a and 3b show the surgical tube 9 of the proctoscope 1 according to the invention in a perspective view in FIG. 3a and in a side view in FIG. 3b.

The surgical tube 9 has a cylindrical shape or a cylindrically conical shape, respectively, and is designed as an elongated, hollow body. The first end 12 of the surgical tube 9 is designed in the shape of a truncated cone or a hemisphere shape and is partially closed. An elongated opening, referred to as the surgical window 13, is disposed at this first end 12 essentially laterally or essentially along a cylinder wall of the surgical tube 9.

The surgical window 13 extends, for example, over a third of the total length of the surgical tube 9, starting at the first end 12 of the surgical tube. The surgical window 13 is designed to be almost rectangular and is dimensioned in such a way that it is possible for a surgeon to carry out a surgical procedure on the patient's tissue protruding in the surgical window.

The second end 14 arranged opposite the first end 12 of the surgical tube 9 has an opening which essentially corresponds to the diameter of the cylindrical surgical tube 9 at the second end 14. This opening can also be designed to be slightly larger than the diameter of the cylindrical surgical tube 9 at the second end 14.

At this second end 14, the surgical tube 9 is designed, for example, with corresponding holes or dimples 15 along the circumference of the second end 14 of the surgical tube 9 in such a way that it can be securely gripped and held by a surgeon. Furthermore, the first alignment marker 16 or a thread attachment 16 is arranged at this second end 14.

To temporarily lock the surgical tube 9 with the fastening ring 2, a second locking element 17 is arranged at the second end 14 of the surgical tube 9. This second locking element 17 is designed in such a way that it can be latched with one of the first locking elements 8 of the fastening ring 2. Such temporary locking can be achieved with a so-called bayonet lock, with which, as is known, a quick and detachable mechanical connection can be established between two cylindrical parts along their longitudinal axis. Depending on which of the first locking elements 8 of the fastening ring 2 latches into or forms a positive connection with the second locking element 17, the surgical tube 9 is fixed in one of the 7 selectable positions (1 o'clock, 3 o'clock, 5 o'clock, 7 o'clock, 9 o'clock, 11 o'clock and 12 o'clock).

At the second end 14 of the surgical tube 9, a radial fixation 18 is arranged for an obturator (not shown in FIGS. 3a and 3b), which can be introduced into the surgical tube 9 and locked therewith.

FIGS. 4a and 4b show two different perspective views of an obturator 19 of the proctoscope 1 according to the invention.

The obturator 19 has a substantially cylindrical or cylindrically conical, longitudinally elongated shape. The obturator 19 has at its first end 20 a conical or hemispherical closure. The obturator 19 is designed in such a way that it can be introduced into the surgical tube 9 and thus completely closes the surgical window 13 of the surgical tube 9. For this purpose, the outer diameter of the obturator 19 is optimally adapted to the inner diameter of the surgical tube 9 and the first end 20 of the obturator 19 is optimally adapted to the first end 12 of the surgical tube 9 having the conical or hemispherical shape.

The second end of the obturator is designed, for example, with corresponding holes or dimples 15 along the circumference of the second end 21 of the obturator 19, allowing it such to be securely grasped and held by a surgeon.

In addition, the second end 21 of the obturator 19 has a further radial fixation 22, with which the obturator 19 can be positively connected to the surgical tube 9. In addition, the second end 21 of the obturator 19 has a second alignment marker 23.

FIG. 5 shows the obturator 19 inserted into the surgical tube 9.

In preparation for the use of the proctoscope 1 in the rectum of a human, the proctoscope 1, consisting of its fastening ring 2, the surgical tube 9 and the obturator 19, is assembled in a first step, whereby the parts are connected or fixed together accordingly.

For this purpose, the obturator 19 is inserted into the surgical tube 9, whereby it is important to ensure that the further radial fixation 22 of the obturator completely engages or is positively connected to the radial fixation 18 of the surgical tube and that the obturator 19 is completely inserted into the surgical tube 9.

This alignment between obturator 19 and surgical tube 9 is achieved when the second alignment marker 23 of the obturator 19 has been aligned with the first alignment marker 16 or the thread attachment 16 of the surgical tube 9, as can be seen in FIG. 5.

The surgical tube 9 with the inserted obturator 19 is then inserted into the fastening ring 2 and the second locking element 17 of the surgical tube 9 is connected or fixed in a form-fitting manner to any of the seven first locking elements 8 of the fastening ring 2. The surgical tube 9 with the inserted obturator 19 is thus fixed and locked in the fastening ring 2.

After the proctoscope 1 has been wetted with a lubricant, it can be inserted into the patient. It should be noted that the handle 5 of the fastening ring 2 points towards 6 o'clock.

After the proctoscope has been inserted, it is fixed via skin sutures through the fixing holes 7 at 12 o'clock, 5 o'clock and 7 o'clock.

If the obturator 19 has been inserted into the surgical tube 9 in this way, the surgical window 13 of the surgical tube 9 is closed by the obturator 19.

FIG. 6 shows the assembled proctoscope 1 according to the invention.

As already explained, in preparation for using the proctoscope 1 in the rectum of a human, the unit consisting of the surgical tube 9 and the inserted and fixed obturator 19 is inserted into the fastening ring 2 and fixed in the manner of a bayonet lock, for example by means of one of the first locking elements 8 arranged on the fastening ring 2 and the second locking element 17 attached to the surgical tube 9. The process of inserting the unit consisting of the surgical tube 9 and the fixed obturator 19 into the fastening ring 2 is illustrated by the first arrow 24. The process of fixing and locking the unit in the fastening ring 2 is illustrated by the second arrow 25.

The three-part proctoscope 1 is shown in FIG. 6 in its assembled form and, for example, in the form introduced into the rectum. In this assembled form, the first end 12 of the surgical tube 9 is arranged at the first end 20 of the obturator 19 and the second end 14 of the surgical tube 9 is arranged at the second end 21 of the obturator 19.

In this assembled form, the positioning aid 11, the first alignment marker 16 or the thread attachment 16 and the second alignment marker 23 are arranged along an imaginary line. Such an imaginary line runs parallel to a longitudinal axis of the surgical tube 9 and/or the obturator 19.

In this assembled form, the surgical window 13 of the surgical tube 9 is completely closed by the obturator 19. The closed surgical window 13 enables improved, friction-reduced insertion of the proctoscope 1, whereby the muscular wall of the rectum is stretched, but the mucous membrane is not damaged.

Representations of the proctoscope 1 in the area of the human rectum have been omitted from the figures, as this is sufficiently known to the person skilled in the art.

FIGS. 7a and 7b show further views of the proctoscope.

For a better understanding, the proctoscope 1 already shown in FIG. 6 is shown in FIG. 7a in a side view and in FIG. 7b in a further perspective view. All elements shown in FIGS. 7a and 7b have already been described in detail in conjunction with the previous figures, so that a repetition at this point is not necessary.

To perform a surgical procedure, the obturator 19 is subsequently removed from the surgical tube 9 arranged and fixed in the fastening ring 2, which is illustrated in FIG. 8 by the third arrow 26. A redundant explanation of the known elements shown in FIG. 8 is omitted.

The surgical window 13 of the surgical tube 9 is now exposed and the surgeon can perform a surgical procedure in the area of the human rectum. This can be done at the location described as the 12 o'clock position, since the surgical window 13 is aligned as shown in FIGS. 6 to 8.

To select a different direction or position for the surgical window 13, the obturator 19 is reinserted into the surgical tube 9, as illustrated by the fourth arrow 27 in FIG. 9. In doing so, the second alignment marker 23 of the obturator 19 is brought into alignment with the first alignment marker 16 of the surgical tube 9.

This insertion of the obturator 19 into the surgical tube 9 completely closes the surgical window 13 in the surgical tube 9. As a result of this insertion, the first alignment marker 16 of the surgical tube 9 and the second alignment marker 23 of the obturator 19 are brought into alignment.

When the obturator 19 is inserted into the surgical tube 9 and the surgical window 13 is thus closed, the surgical tube 9 and thus of the surgical window 13 can be safely rotated to different positions.

To change the position of the surgical window 13, the unit consisting of the surgical tube 9 with the obturator 19 inserted is rotated in the direction indicated by the arrow 28 in FIG. 10 and then pulled out of the fastening ring 2, as indicated by the arrow 29. This process corresponds to the release or opening of the bayonet lock, which is formed by the first locking element 8 (not shown in FIG. 10) and the second locking element 17.

The surgical tube 9 with the obturator 19 inserted is then rotated into a position where it is completely pulled out of the fastening ring 2. The rotation process is indicated in FIG. 11 by the arrow 30, whereby in the selected illustration the surgical tube 9 with the obturator 19 inserted is not shown as having been completely pulled out of the fastening ring 2. Rotating the surgical tube 9 in the anal canal must be avoided at all costs, as this could otherwise lead to torsion of the tissue.

In the example of FIG. 11, the surgical window 13 is rotated to the 3 o'clock position. For this purpose, the first alignment marker 16 and the second alignment marker 23 are brought into alignment with the 3 o'clock position marker 10 shown in FIG. 11 with the reference number 10.

To fix the surgical window 13 in this 3 o'clock position, the surgical tube 9 with the obturator 19 inserted is pressed into the fastening ring 2 and rotated, whereby the bayonet lock is locked again in the new 3 o'clock position. This process is a reversal of the sequence described in FIG. 10 and is not shown separately.

The other positions 10, 11 can be reached or selected for the alignment of the surgical window 13 in the same manner.

Moreover, the part of the system that can rotate in the fastening ring 2, i.e. the surgical tube 9, can be locked and thus fixed by turning in, for example, 7 positions. These positions correspond to the position markers 10 visible in FIG. 1 at the positions 1 o'clock, 3 o'clock, 5 o'clock, 7 o'clock, 9 o'clock, 11 o'clock and 12 o'clock. Another position is the 12 o'clock mark on the fastening ring.

To perform a surgical procedure, the obturator 19 is subsequently removed from the surgical tube 9 which is arranged and fixed in the fastening ring 2, as illustrated in FIG. 8 by the third arrow 26. In this way, the surgical window is released at the newly selected position.

To remove the proctoscope 1, the obturator 19 is reinserted into the surgical tube 9, as illustrated by the fourth arrow 27 in FIG. 9. In doing so, the second alignment marker 23 of the obturator 19 is brought into alignment with the first alignment marker 16 of the surgical tube 9.

This insertion of the obturator 19 into the operating tube 9 once again completely closes the surgical window 13 in the operating tube 9.

Subsequently, for example, the surgical tube 9 with the obturator 19 inserted is removed from the fastening ring 2 by opening the bayonet lock, as described with reference to FIG. 10. Subsequently, the surgical tube 9 with the obturator 19 inserted is completely pulled out of the fastening ring 2.

The fastening ring 2 is then released by cutting the skin sutures and removed from the area of the human rectum.

FIG. 12 shows the assembled proctoscope 1 and FIG. 13 shows an enlargement of the area surrounded by a ring in FIG. 12 of the radial fixation 18 on the surgical tube 9 and the further radial fixation 22 on the obturator 19.

FIG. 12 shows the three-part proctoscope 1 already known from FIG. 6 in its assembled form. The proctoscope 1 includes the fastening ring 2, the surgical tube 9 and the obturator 19. In an area of the proctoscope 1 that is surrounded by a ring in FIG. 12, the radial fixation 18 of the surgical tube 9 and the further radial fixation 22 of the obturator 19 are shown.

FIG. 13 shows that the further radial fixation 22 is designed as a protrusion on the obturator 19 and is U-shaped, with arcuate locking protrusions 32 being arranged on opposite parallel outer legs 31 of the U-shaped further radial fixation 22. Furthermore, FIG. 13 shows that the radial fixation 18 is designed as a U-shaped notch in the surgical tube 9, with arcuate locking depressions 34 being arranged on opposite parallel inner legs 33 of the radial fixation 18.

The arched locking protrusions 32 and the arched locking depressions 34 are designed to correspond with one another. They therefore have complementary shapes and allow the arched locking protrusions 32 to snap into the arched locking depressions 34 when the obturator 19 is inserted into the surgical tube 9, thus forming a positive connection between the obturator 19 and the surgical tube 9.

### LIST OF REFERENCE SYMBOLS

1 proctoscope
2 fastening ring
3 first area of fastening ring (cylindrical)
4 second area of fastening ring (flange)
5 handle
6 grip recess
7 fastening holes
8 first locking elements
9 surgical tube
10 position markers
11 positioning aid/12 o'clock mark
12 first end of the surgical tube
13 surgical window
14 second end of the surgical tube
15 holes/dimples
16 first alignment marker/thread attachment
17 second locking element
18 radial fixation on the surgical tube for obturator
19 obturator
20 first end of the obturator
21 second end of the obturator
22 further radial fixation
23 second alignment marker
24 first arrow
25 second arrow
26 third arrow
27 fourth arrow
28 fifth arrow
29 sixth arrow
30 seventh arrow
31 outer leg
32 arched locking protrusions
33 inner leg
34 arched locking recesses

1.-10. (canceled)

11. A proctoscope comprising three parts, having:
a first part constructed as a securing ring having open opposite sides and first region having an at least partially cylindrical shape or cylindrically conical shape and a flange-shaped second region, wherein the securing ring is composed of a transparent material, has several position markers, a positioning aid in the form of a 12 o'clock mark and several first locking elements,
a second part constructed as a surgical tube configured to be disposed in an inner diameter of the securing ring and has a second locking element that can be positively connected and fixed to one of the first locking elements, the surgical tube having a frustoconical or hemispherical, at least partially closed first end and a surgical window shaped as a rectangular opening arranged essentially along a cylinder wall of the surgical tube, and a second end having at least one first alignment marker with a thread attachment and a radial fixation, and
a third part constructed as an obturator designed to be inserted and radially fixed in the surgical tube, the obturator having a first conical or hemispherical closed end adapted to the first end of the surgical tube and completely closing the surgical window when inserted and radially fixed in the surgical tube, and that a further radial fixation arranged so as to snap into the radial fixation disposed on the surgical tube when the obturator is inserted into the surgical tube.

12. The proctoscope of claim 11, wherein the securing ring comprises a plurality of fastening holes and a handle.

13. The proctoscope of claim 11, wherein the further radial fixation is U-shaped and has arcuate locking protrusions arranged on opposite parallel outer legs of the U-shaped further radial fixation, wherein the radial fixation is also U-shaped and has arcuate locking recesses arranged on opposite parallel inner legs of the U-shaped radial fixation, and wherein the arcuate locking protrusions and the arcuate locking recesses have corresponding, complementary shapes and are configured to engage with one another when the obturator is inserted in the surgical tube.

14. The proctoscope of claim 11, wherein a plurality of holes or dimples are arranged at the second end of the surgical tube along the circumference of the surgical tube.

15. The proctoscope of claims 11, wherein the obturator has a second alignment marker at its second end and a plurality of holes or dimples arranged along the circumference of the surgical tube.

16. A method of using a proctoscope, the proctoscope comprising three parts and the proctoscope is used in insertion into a rectum of a patient, having
as a first part, a securing ring having open opposite sides with a first region with an at least partially cylindrical shape or cylindrically conical shape and a flange-shaped second region;
as a second part, a surgical tube; and,
as a third part, an obturator,
the method comprising the steps of:
introducing and securing the securing ring in the rectum,
introducing and radially fixing the obturator in the surgical tube so as to form a unit,
introducing and locking the unit consisting of the surgical tube and the obturator in the securing ring,
removing the obturator from the surgical tube and releasing a surgical window in the surgical tube, which enables a surgical intervention in the area of the rectum,
removing the proctoscope by introducing and locking the obturator in the surgical tube,
releasing and removing the unit consisting of the surgical tube with the introduced obturator from the securing ring, and
releasing and removing the securing ring,
wherein for locking the surgical tube with the obturator introduced into the securing ring, several first locking elements are provided in the securing ring and a second locking element is provided on the surgical tube, wherein one of the first locking elements can be connected to and fixed in a form-fitting manner with the second locking element, and
wherein for a positive connection of the obturator introduced into the surgical tube with the surgical tube, a radial fixation is provided on the surgical tube and a further radial fixation is provided on the obturator, which enable the positive connection to snap into place by engaging with one another.

17. The method of claim 16, wherein the unit formed of the surgical tube and the inserted obturator is introduced into the securing ring and rotated before locking with the securing ring, until a position of a first alignment marker of the surgical tube has been brought into alignment with the position of a position marker or a positioning aid provided on the securing ring, wherein the surgical window of the surgical tube is aligned in the direction shown by the position marker or the positioning aid.

18. The method of claim 16, wherein the securing ring has six position markers and a positioning aid, with the position markers having orientations 1 o'clock, 3 o'clock, 5 o'clock, 7 o'clock, 9 o'clock, 11 o'clock and the positioning aid has an orientation 12 o'clock.

19. The method of claim 16, wherein the further radial fixation is U-shaped, wherein arcuate locking protrusions are provided on opposite parallel outer legs of the U-shaped further radial fixation, wherein the radial fixation is also U-shaped, wherein arcuate locking recesses are provided on opposite parallel inner legs of the radial fixation, and wherein the arcuate locking protrusions and the arcuate locking recesses have mutually corresponding, complementary shapes and thus engage with one another when the obturator is inserted into the surgical tube and produce a positive connection.

20. The method of claim 16, wherein the securing ring is composed of a transparent material.
